(19) United States
(12) Patent Application Publication
(10) Pub. No.: US 2023/0172598 A1
(43) Pub. Date: Jun. 8, 2023

(54) VASCULAR CLOSURE DEVICES AND METHODS

(71) Applicant: MAYO FOUNDATION FOR MEDICAL EDUCATION AND RESEARCH, Rochester, MN (US)

(72) Inventor: Rabih G. Tawk, Ponta Vedra, FL (US)

(21) Appl. No.: 18/073,819

(22) Filed: Dec. 2, 2022

Related U.S. Application Data
(60) Provisional application No. 63/285,154, filed on Dec. 2, 2021.

(51) Int. Cl. A61B 17/00

(52) U.S. Cl. CPC A61B 17/0057; A61B 2017/00601; A61B 2017/00619; A61B 2017/00592

(57) ABSTRACT

Vascular closure devices along with systems and methods of delivery for deploying the vascular closure devices are described herein.

### RELATED APPLICATION

This application claims the benefit under 35 U.S.C. Section 119 of U.S. Provisional Patent Application Ser. No. 63/285,154 entitled “VASCULAR CLOSURE DEVICE AND METHOD” and filed on Dec. 2, 2021, which is incorporated herein by reference in its entirety.

Vascular closure devices along with systems and methods of delivery for deploying the vascular closure devices are described herein.

Known vascular closure devices for use during endovascular transfemoral thrombectomies can result in failed procedures, loss of neurons, and loss of life, due to long procedure times and failure to remove blood clots. Such devices for use during endovascular transcarotid thrombectomies can fail to close the common carotid artery safely and effectively.

### SUMMARY

Vascular closure devices that have an inner layer, outer layer, and optional sealing component, and systems and methods for delivering the vascular closure devices are described herein.

The vascular closure devices described herein may be useful in closing blood vessels used for accessing a patient's vasculature in procedures such as, e.g., endovascular transcarotid thrombectomies, where standard vascular closure devices can fail to close the common carotid artery safely and effectively. In one or more embodiments, the vascular closure devices may provide localized compression around vascular openings in which the vascular closure devices are deployed.

In a third aspect, one or more embodiments of the vascular closure devices described herein may include an inner layer which may include an intravascular tapered body and an anchor attached to a first base of the intravascular tapered body; wherein the intravascular tapered body may have an interior channel extending through the intravascular tapered body along a channel axis from a first apex of the intravascular tapered body towards the first base of the intravascular tapered body, wherein the interior channel may extend from an aperture proximate the first apex to a first end located between the first base and the aperture, wherein the anchor may have a delivery configuration and a deployed configuration, wherein the anchor may have an anchor width measured transverse to the channel axis, wherein the anchor width when the anchor is in the deployed configuration may be greater than the anchor width when the anchor is in the delivery configuration, wherein the anchor may be configured for location within a blood vessel when in the deployed configuration, wherein the first apex may be configured to protrude outside of the blood vessel in the deployed configuration; and the vascular closure devices may have an outer layer which may include an extravascular tapered body and a male locking component which may include an interior shaft, wherein the extravascular tapered body may comprise a second apex and a second base, wherein the interior shaft may extend from the second apex to towards the second base to a second end located between the second base and the second apex, wherein the interior shaft may be configured for insertion into the interior channel; and the vascular closure devices may have an optional sealing component which may be located between the intravascular tapered body and at least a portion of an interior of the extravascular tapered body; wherein the intravascular tapered body may be located within the extravascular tapered body when the male locking component may be located in the interior channel of the intravascular tapered body.

In some embodiments of the vascular closure devices described herein, the interior shaft may further include a through-hole which may be aligned with the channel axis when the male locking component is located in the interior channel of the intravascular tapered body. In still other embodiments, the interior shaft may form an interference fit with the interior channel when the interior shaft is inserted into the interior channel. In still other embodiments, the interior channel may further include a constriction nearer to the first apex than the first end. In still other embodiments, the interior shaft may include at least one bulbous component.

In some embodiments of the vascular closure devices described herein, the anchor may include one or more extensions extending away from the channel axis and the first base of the intravascular tapered body when the anchor may be in the deployed configuration. In still other embodiments, the anchor is detached or detachable from the first base of the intravascular tapered body.

In some embodiments of the vascular closure devices described herein, the first end is open. In still other embodiments, the first end is closed. In still other embodiments, the first end is located beyond both the aperture and the first base.

In some embodiments of the vascular closure devices described herein, the vascular closure device may be configured to be delivered into the blood vessel through a delivery lumen a delivery device.

In some embodiments of the vascular closure devices described herein, the first base of the intravascular tapered body may include a circular first base. In still other embodiments, the second base of the extravascular tapered body may include a circular second base.

In some embodiments of the vascular closure devices described herein, the sealing component may be made up of collagen or any collagen-like substance. In still other embodiments, the sealing component may be expandable or deformable to seal the blood vessel.

In some embodiments, the vascular closure devices described herein may include an inner layer comprising an intravascular tapered body, an anchor attached to a first lower base of the intravascular tapered body, and a bulbous top which may be attached to an upper end of the intravascular tapered body, wherein the anchor may comprise a delivery configuration and a deployed configuration, wherein the anchor may comprise an anchor width measured transverse to an axis extending through the bulbous body and the first lower base, wherein the anchor width when the anchor is in the deployed configuration may be greater than the anchor width when the anchor is in the delivery configuration, wherein the anchor may be configured for location within a blood vessel when in the deployed configuration, wherein the intravascular tapered body and the bulbous top may be configured to protrude outside of the blood vessel when the anchor is in the deployed configuration in the blood vessel; and the vascular closure devices may have an outer layer which may comprise an extravascular tapered body, wherein the extravascular tapered body may comprise an upper aperture and a second lower base, wherein the extravascular tapered body may be configured for placement onto the intravascular tapered body such that the bulbous top of the intravascular tapered body may protrude from the upper aperture of the extravascular tapered body; and the vascular closure devices may have an optional sealing component coating between the intravascular tapered body and at least a portion of an interior of the extravascular tapered body; wherein the intravascular tapered body may be located within the extravascular tapered body when the bulbous top protrudes from the upper aperture of the extravascular tapered body.

In some embodiments of the vascular closure devices described herein, the upper aperture of the extravascular tapered body may form an interference fit with the bulbous top of the intravascular tapered body when the intravascular tapered body is located within the extravascular tapered body such that the bulbous top may protrude from the upper aperture of the extravascular tapered body. In still other embodiments, a width of the bulbous top of the intravascular tapered body measured transverse to the axis may be greater than a width of the upper aperture of the extravascular tapered body. In still other embodiments, the anchor is detached or detachable from the first base of the intravascular tapered body. In still other embodiments, the sealing component may be expandable or deformable to fully seal the blood vessel.

In some embodiments of the vascular closure devices described herein, the first lower base of the intravascular tapered body may comprise a circular base. In still other embodiments, the second lower base of the extravascular tapered body may comprise a circular base.

In some embodiments of the vascular closure devices described herein, the intravascular tapered body may be located within the extravascular tapered body such that the second lower base of the extravascular tapered body may be located adjacent to the first lower base of the intravascular tapered body and the upper aperture of the extravascular tapered body may be located adjacent to the upper end of the intravascular tapered body in the deployed configuration.

In some embodiments, the vascular closure devices described herein may include an inner layer which may comprise an intravascular tapered body with a central axis and an anchor which may be attached to a first base of the intravascular tapered body, wherein the anchor may comprise a delivery configuration and a deployed configuration, wherein the anchor may comprise an anchor width measured transverse to the central axis, wherein the anchor width when the anchor is in the deployed configuration may be greater than the anchor width when the anchor is in the delivery configuration, wherein the anchor may be configured for location within a blood vessel when in the deployed configuration; and the vascular closure devices may have an outer layer which may comprise an extravascular tapered body; and the vascular closure devices may have an optional sealing component coating which may be located between the intravascular tapered body and at least a portion of an interior of the extravascular tapered body; wherein the intravascular tapered body may be located within the extravascular tapered body in the deployed configuration.

In some embodiments, the vascular closure device described herein may include an inner layer comprising an intravascular tapered body with a central axis and an anchor attached to a first base of the intravascular tapered body; and the vascular closure devices may have an outer layer comprising an extravascular tapered body; and the vascular closure devices may have an optional sealing component coating which may be located between the intravascular tapered body and at least a portion of an interior of the extravascular tapered body; wherein the intravascular tapered body may be located within the extravascular tapered body after the vascular closure device is deployed within a blood vessel.

In some embodiments, the vascular closure device delivery systems described herein may include a vascular closure device that may include an inner layer which may comprise an intravascular tapered body, an anchor attached to a first base of the intravascular tapered body, wherein the intravascular tapered body may comprise an interior channel extending through the intravascular tapered body along a channel axis from a first apex of the intravascular tapered body towards the first base of the intravascular tapered body, wherein the interior channel may extend from an aperture proximate the first apex to a first end located between the first base and the aperture; and an outer layer which may comprise an extravascular tapered body and a male locking component comprising an interior shaft, wherein the extravascular tapered body may comprise a second apex and a second base, wherein the interior shaft may extend from the second apex to towards the second base to a second end which may be located between the second base and the second apex, wherein the interior shaft may be configured for insertion into the interior channel; and an optional sealing component which may be located between the intravascular tapered body and at least a portion of an interior of the extravascular tapered body; wherein the anchor may comprise a delivery configuration and a deployed configuration, wherein the anchor may comprise an anchor width measured transverse to the channel axis, wherein the anchor width when the anchor is in the deployed configuration may be greater than the anchor width when the anchor is in the delivery configuration, wherein the anchor may be configured for location within a blood vessel when in the deployed configuration, wherein the first apex may be configured to protrude outside of the blood vessel in the deployed configuration, wherein the intravascular tapered body may be located within the extravascular tapered body when the male locking component is located in the interior channel of the intravascular tapered body.

In some embodiments of the vascular closure device delivery systems described herein, the first end is open. In still other embodiments, the first end is closed. In still other embodiments, the first end is located beyond both the aperture and the first base.

In some embodiments of the vascular closure device delivery systems described herein, the delivery configuration may comprise placing the vascular closure device perpendicular to the deployed configuration. In still other embodiments, the delivery configuration may comprise placing the vascular closure device parallel to the deployed configuration.

In some embodiments of the vascular closure device delivery systems described herein, the anchor is detached or detachable from the first base of the intravascular tapered body.

In some embodiments of the vascular closure device delivery systems described herein, the sealing component may be expandable or deformable to fully seal the blood vessel.

In some embodiments, the vascular closure device delivery systems described herein may include a vascular closure device that may include an inner layer which may comprise an intravascular tapered body, an anchor which may be attached to a first lower base of the intravascular tapered body, and a bulbous top which may be attached to an upper end of the intravascular tapered body; and an outer layer which may comprise an extravascular tapered body, wherein the extravascular tapered body may comprise an upper aperture and a second lower base; and an optional sealing component coating which may be located between the intravascular tapered body and at least a portion of an interior of the extravascular tapered body; wherein the anchor may comprise a delivery configuration and a deployed configuration, wherein the anchor may comprise an anchor width measured transverse to an axis extending through the bulbous body and the first lower base, wherein the anchor width when the anchor is in the deployed configuration may be greater than the anchor width when the anchor is in the delivery configuration, wherein the anchor may be configured for location within a blood vessel when in the deployed configuration, wherein the intravascular tapered body and the bulbous top may be configured to protrude outside of the blood vessel when the anchor is in the deployed configuration in the blood vessel, wherein the extravascular tapered body may be configured for placement onto the intravascular tapered body such that the bulbous top of the intravascular tapered body may protrude from the upper aperture of the extravascular tapered body, wherein the intravascular tapered body may be located within the extravascular tapered body when the bulbous top protrudes from the upper aperture of the extravascular tapered body.

In some embodiments of the vascular closure device delivery systems described herein, the delivery configuration may comprise placing the vascular closure device perpendicular to the deployed configuration. In still other embodiments, the delivery configuration may comprise placing the vascular closure device parallel to the deployed configuration.

In some embodiments of the vascular closure device delivery systems described herein, the anchor is detached or detachable from the first base of the intravascular tapered body.

In some embodiments of the vascular closure device delivery systems described herein, the sealing component may be expandable or deformable to fully seal the blood vessel.

In some embodiments, the methods described herein may include a method of implanting a vascular closure device that includes positioning the anchor of a vascular closure device as described herein in a blood vessel, wherein the positioning occurs while the anchor of the intravascular tapered body is in the delivery configuration; and expanding the anchor from the delivery configuration to the deployed configuration after positioning the anchor in the blood vessel; and positioning the optional sealing component over the intravascular tapered body; and positioning the extravascular tapered body over the intravascular tapered body and the optional sealing component after positioning the anchor in the blood vessel, wherein positioning the extravascular tapered body over the intravascular tapered body and the optional sealing component comprises inserting the interior shaft of the extravascular tapered body into the interior channel of the intravascular tapered body, wherein the extravascular tapered body compresses the optional sealing component to fully seal the blood vessel and maintain such compression.

In some embodiments, the methods described herein may include a method of implanting a vascular closure device that includes positioning the anchor of the intravascular tapered body of a vascular closure device as described herein in a blood vessel, wherein the positioning occurs while the anchor of the intravascular tapered body is in the delivery configuration; and expanding the anchor from the delivery configuration to the deployed configuration after positioning the anchor in the blood vessel; and positioning the extravascular tapered body over the intravascular tapered body after positioning the anchor in the blood vessel, wherein positioning the extravascular tapered body over the intravascular tapered body may comprise placing the extravascular tapered body over the bulbous top of the intravascular tapered body and onto the intravascular tapered body, such that the bulbous top may protrude beyond the upper aperture of the extravascular tapered body.

In some embodiments, a vascular closure device as described herein includes: a cone-shaped inner component extending from a base to an apex along a device axis extending through the base and the apex; an outer component defining a passageway extending along the device axis from an anchor portion proximate the base of the inner component to a collar proximate the apex of the inner component, wherein the anchor portion of the outer component is configured to expand radially relative to the device axis when the inner component advances through the passageway of the outer component; and a locking element attached to the inner component proximate the apex of the inner component, wherein the locking element is configured to form a mechanical interlock with the collar of the outer component to resist movement of the inner component out of the passageway of the outer component.

In some embodiments of the vascular closure devices described herein, the outer component comprises a plurality of ribs extending from the collar towards the base of the inner component and wherein the anchor portion comprises a plurality of anchor feet extending away from the device axis, wherein each rib of the plurality of ribs comprises one anchor foot of the plurality of anchor feet, wherein the plurality of anchor feet collectively define the anchor portion of the outer component.

In some embodiments of the vascular closure devices described herein, the device comprises a tensioning element attached to the locking element and the inner component, the tensioning element extending through the collar of the outer component, and the tensioning element configured to draw the locking element through the collar when a tension force is applied to the locking element through the tensioning element. In some embodiments, the collar comprises an assembly slot configured to allow passage of the tensioning element during assembly of the inner component and the outer component. In some embodiments, the tensioning element comprises suture material.

In some embodiments of the vascular closure devices described herein, the locking element defines the apex of the inner component. In some embodiments, the inner component comprises a neck located between the locking element a remainder of the inner component.

In some embodiments of the vascular closure devices described herein, the locking element is attached to the apex of the inner component by a tensioning element extending from the apex of the inner component and through the locking element and the collar of the outer component, wherein a tension force applied to the tensioning element draws the locking element into the mechanical interlock with the collar of the outer component. In some embodiments, the apex of the inner component is spaced apart from the locking element such that the collar of the outer component is positioned between the locking element and the apex of the inner component. In some embodiments, the tensioning element is in tension when the locking element is in the mechanical interlock with the collar of the outer component.

In some embodiments of the vascular closure devices described herein, the device comprises a seal component that comprises a seal ring attached to a tubular seal, wherein the seal ring is configured forms a second mechanical interlock with the collar when the tubular seal is positioned over the outer component, wherein the second mechanical interlock between the seal ring and the collar resists movement of the seal ring away from the anchor portion of the outer component. In some embodiments, the collar of the outer component is positioned between the locking element and the seal ring when the locking element forms the mechanical interlock with the collar and the seal ring forms the second mechanical interlock with the collar. In some embodiments, the tubular seal conforms to an external shape of the outer component when the seal ring forms the second mechanical interlock with the collar.

In some embodiments, a vascular closure device as described herein includes: a cone-shaped inner component extending from a base to an apex along a device axis extending through the base and the apex; an outer component defining a passageway extending along the device axis from an anchor portion proximate the base of the inner component to a collar proximate the apex of the inner component, wherein the anchor portion of the outer component is configured to expand radially relative to the device axis when the inner component advances through the passageway of the outer component; a locking element attached to the inner component proximate the apex of the inner component, wherein the locking element is configured to form a mechanical interlock with the collar of the outer component to resist movement of the inner component out of the passageway of the outer component; a tensioning element attached to the locking element and the inner component, the tensioning element extending through the collar of the outer component, the tensioning element configured to draw the locking element through the collar when a tension force is applied to the locking element through the tensioning element; and a seal component that comprises a seal ring attached to a tubular seal, wherein the seal ring is configured forms a second mechanical interlock with the collar when the tubular seal is positioned over the outer component, wherein the second mechanical interlock between the seal ring and the collar resists movement of the seal ring away from the anchor portion of the outer component.

In some embodiments of the vascular closure devices described herein, the locking element is attached to the apex of the inner component by a tensioning element extending from the apex of the inner component and through the locking element and the collar of the outer component, wherein a tension force applied to the tensioning element draws the locking element into the mechanical interlock with the collar of the outer component, wherein the apex of the inner component is spaced apart from the locking element such that the collar of the outer component is positioned between the locking element and the apex of the inner component; and wherein the tensioning element is in tension when the locking element is in the mechanical interlock with the collar of the outer component.

In some embodiments of the vascular closure devices described herein, the collar of the outer component is positioned between the locking element and the seal ring when the locking element forms the mechanical interlock with the collar and the seal ring forms the second mechanical interlock with the collar.

In some embodiments of a method of implanting a vascular closure device in a vascular access site as described herein, the method includes: delivering a vascular closure device into a blood vessel through an access site, the vascular closure device comprising: a cone-shaped inner component extending from a base to an apex along a device axis extending through the base and the apex, an outer component defining a passageway extending along the device axis from an anchor portion proximate the base of the inner component to a collar proximate the apex of the inner component, and a locking element attached to the inner component proximate the apex of the inner component. The method further comprises: retracting the collar of the outer component out of the blood vessel through the access site; advancing the locking element and the inner component through the passageway of the outer component, wherein the inner component radially expands the anchor portion of the outer component relative to the device axis, wherein the anchor portion of the outer component is retained in the blood vessel; and mechanically interlocking the locking element with the collar of the outer component to resist movement of the inner component out of the passageway of the outer component.

In some embodiments of the methods of implanting vascular closure devices described herein, advancing the locking element and the inner component comprises applying a tension force to a tensioning element attached to the locking element and the inner component, wherein the tensioning element extends through the collar of the outer component.

In some embodiments of the methods of implanting vascular closure devices described herein, the method includes: advancing a seal ring and a tubular seal of a seal component towards the anchor portion of the outer component along the device axis, wherein the tubular seal deforms to form a seal around the outer component at the access site; and mechanically interlocking the seal ring with the collar of the outer component resists movement of the seal ring and the tubular seal away from the anchor portion of the outer component.

In some embodiments of the methods of implanting vascular closure devices described herein, advancing the locking element and the inner component comprises applying a tension force to a tensioning element attached to the locking element and the inner component, wherein the tensioning element extends through the collar of the outer component, and wherein the method further includes: advancing a seal ring and a tubular seal of a seal component towards the anchor portion of the outer component along the device axis, wherein the tubular seal deforms to form a seal around the outer component at the access site; and mechanically interlocking the seal ring with the collar of the outer component to resist movement of the seal ring and the tubular seal away from the anchor portion of the outer component, wherein mechanically interlocking the seal ring with the collar of the outer component is performed after mechanically interlocking the locking element with the collar of the outer component.

The above summary is not intended to describe each embodiment or every implementation of the present invention. Rather, a more complete understanding of the invention will become apparent and appreciated by reference to the following Description of Exemplary Embodiments and claims in view of the accompanying figures of the drawing.

### BRIEF DESCRIPTION OF THE VIEWS OF THE DRAWING

The present invention will be further described with reference to the figures of the drawing, wherein:

FIG. 1 is a perspective view of one embodiment of an inner layer of a vascular closure device as described herein including directional indicators.

FIG. 2 is a side view of the inner layer of FIG. 1.

FIG. 3 is a perspective view of one embodiment of an outer layer of a vascular closure device as described herein.

FIG. 4 is another perspective view of the outer layer of FIG. 3.

FIG. 5 is a side view of the outer layer of FIG. 3.

FIG. 6 is a perspective view of one embodiment of a vascular closure device include the inner layer of FIG. 1 and the outer layer of FIG. 3.

FIG. 7 is a side view of the vascular closure device of FIG. 6.

FIG. 8 is a perspective view of another embodiment of an inner layer of a vascular closure device as described herein including directional indicators.

FIG. 9 is a side view of the inner layer of FIG. 8.

FIG. 10 is a perspective view of another embodiment of an outer layer of a vascular closure device as described herein.

FIG. 11 is a side view of the outer layer of FIG. 10.

FIG. 12 is a perspective view of another embodiment of a vascular closure device include the inner layer of FIG. 8 and the outer layer of FIG. 10.

FIG. 13 is a side view of the vascular closure device of FIG. 12.

FIG. 14 is a top view of an inner layer of a vascular closure device as described herein.

FIG. 15 is a side view of an inner layer of a vascular closure device as described herein, in a delivery configuration within a delivery device as described herein.

FIG. 16 is a side view of a vascular closure device as described herein, in a delivery configuration within a delivery device as described herein.

FIG. 17 is a side view of a vascular closure device as described herein, in a deployed configuration within a delivery device as described herein.

FIG. 18 is a side view of a vascular closure device as described herein, in a deployed configuration within a blood vessel as described herein.

FIG. 19 is a perspective view of another illustrative embodiment of a vascular closure device as described herein.

FIG. 20 is a cross-sectional view of the vascular closure device depicted in

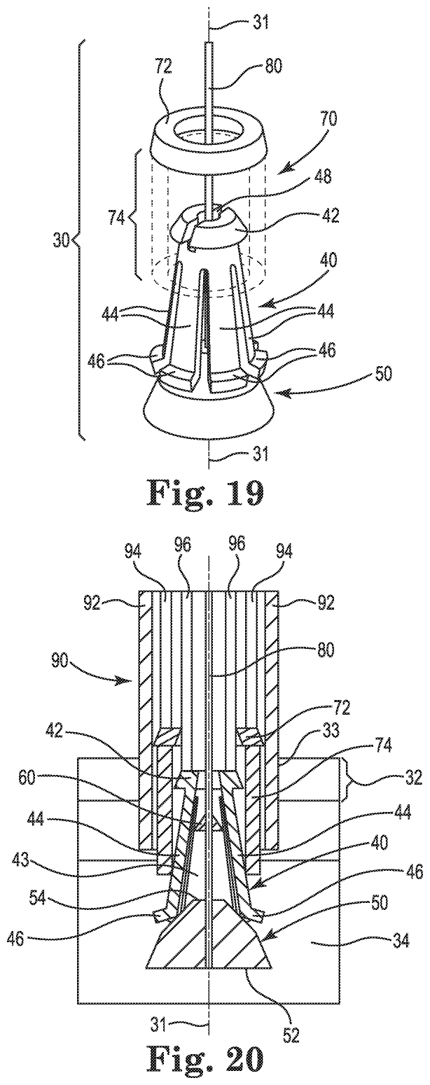

FIG. 19 taken in a plane containing device axis 31 along with the distal end of a delivery apparatus with the distal end of the delivery apparatus and the vascular closure device positioned within a blood vessel through an access site before deployment of the vascular closure device as described herein.

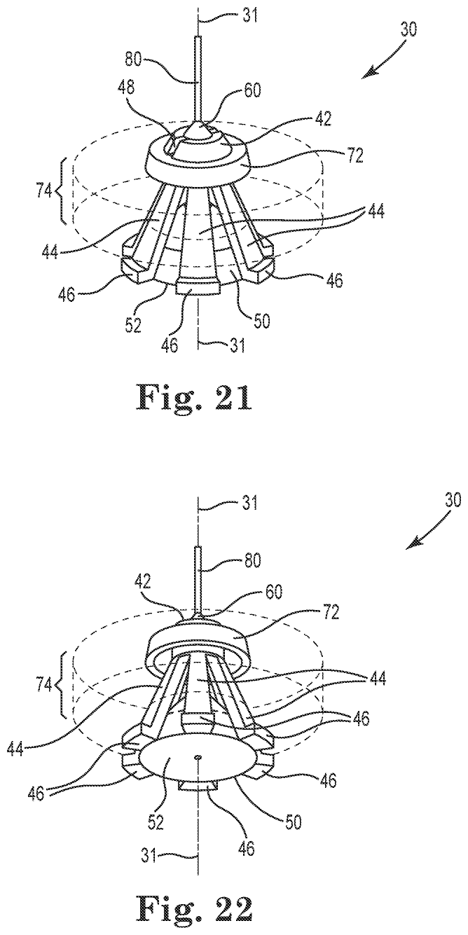

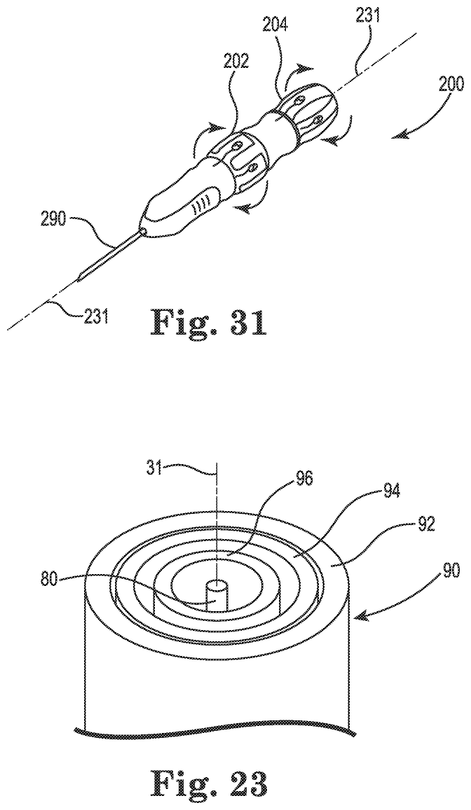

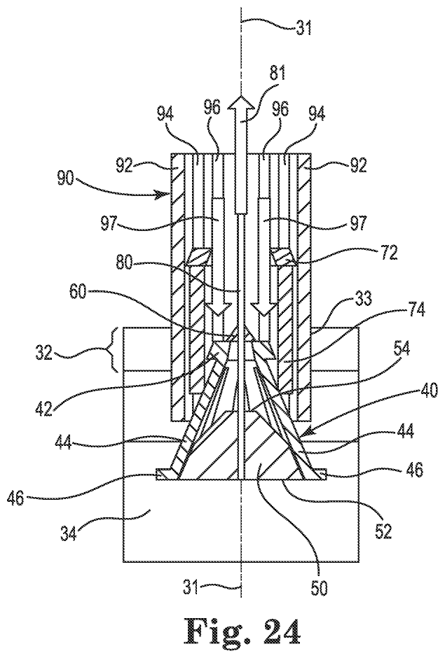

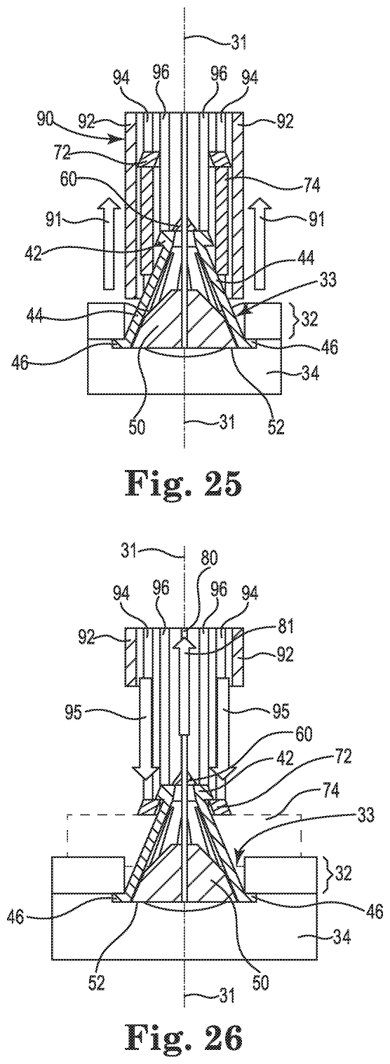

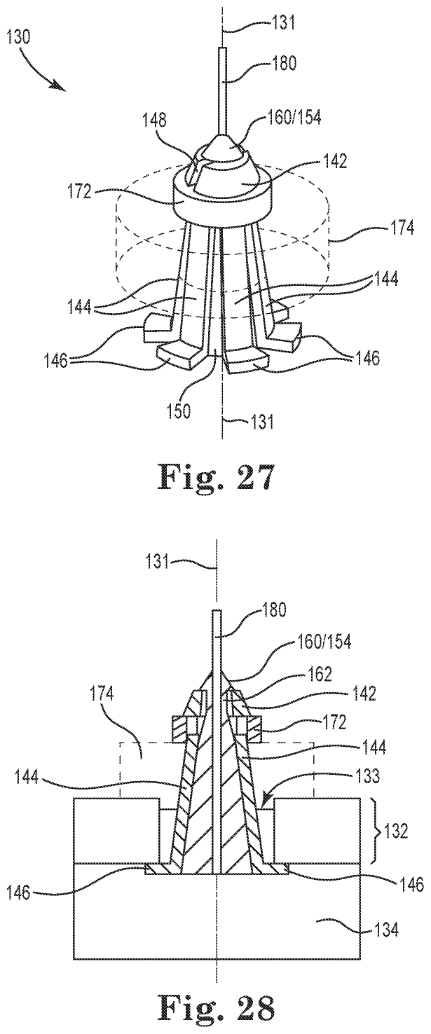

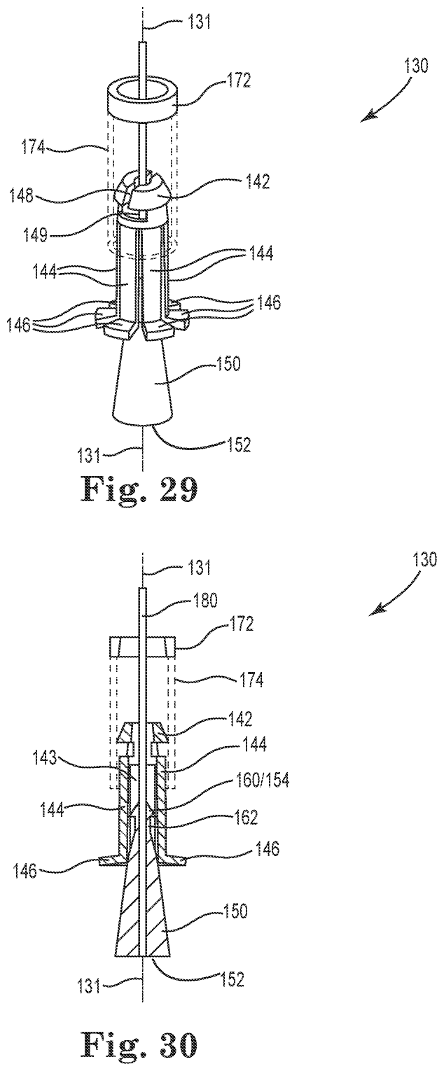

FIG. 21 is a top perspective view of the vascular closure device of FIGS. 19-20 after assembly of the components of the vascular closure device as described herein.

FIG. 22 is a bottom perspective view of the vascular closure device of FIG. 21.

FIG. 23 is a perspective view of an end of the delivery apparatus depicted in the cross-sectional view of FIG. 20.

FIG. 24 is a cross-sectional view of the vascular closure device and delivery apparatus of FIG. 20 at an intermediate step in deployment of the vascular closure device as described herein.

FIG. 25 is a cross-sectional view of the vascular closure device and delivery apparatus of FIGS. 20 and 24 during further deployment of the vascular closure device as described herein.

FIG. 26 is a cross-sectional view of the vascular closure device and delivery apparatus of FIG. 25 after further deployment of the vascular closure device as described herein.

FIG. 27 is a perspective view of another alternative embodiment of a vascular closure device in its deployed state as described herein.

FIG. 28 is a cross-sectional view of the vascular closure device of FIG. 27 after deployment of the vascular closure device in an access site of a blood vessel as described herein.

FIG. 29 is a perspective view of the vascular closure device of FIGS. 27-28 before deployment of the vascular closure device as depicted in FIGS. 27-28.

FIG. 30 is a cross-sectional view of the vascular closure device of FIG. 29 taken in a plane containing device axis 131.

FIG. 31 is a perspective view of one deployment actuation apparatus at the proximal end of a delivery apparatus as described herein.

### DETAILED DESCRIPTION OF ILLUSTRATIVE EMBODIMENTS

In the following detailed description of illustrative embodiments, reference is made to the accompanying figures of the drawing which form a part hereof, and in which are shown, by way of illustration, specific embodiments of the vascular closure devices and delivery systems described herein. It is to be understood that other embodiments may be utilized and structural changes may be made without departing from the scope of the present invention.

The vascular closure devices described herein are sized to safely and effectively close a vascular opening made to access a blood vessel located proximate the skin of a patient such as, e.g., the carotid artery. In one or more embodiments, the vascular closure devices may provide localized compression around vascular openings in which the vascular closure devices are deployed.

The specific constructions used for the vascular closure devices may vary considerably, e.g., the vascular closure devices may be manufactured of any suitable material or combination of materials (e.g., metals, polymers, shape memory materials, absorbent or non-absorbent materials etc.), the vascular closure devices may have one or more anchors, the vascular closure devices may have an anchor that is self-deploying or require a deployment force to deploy the anchor, the vascular closure devices may be self-connecting or require a connection force to connect the outer layer to the inner layer; etc.

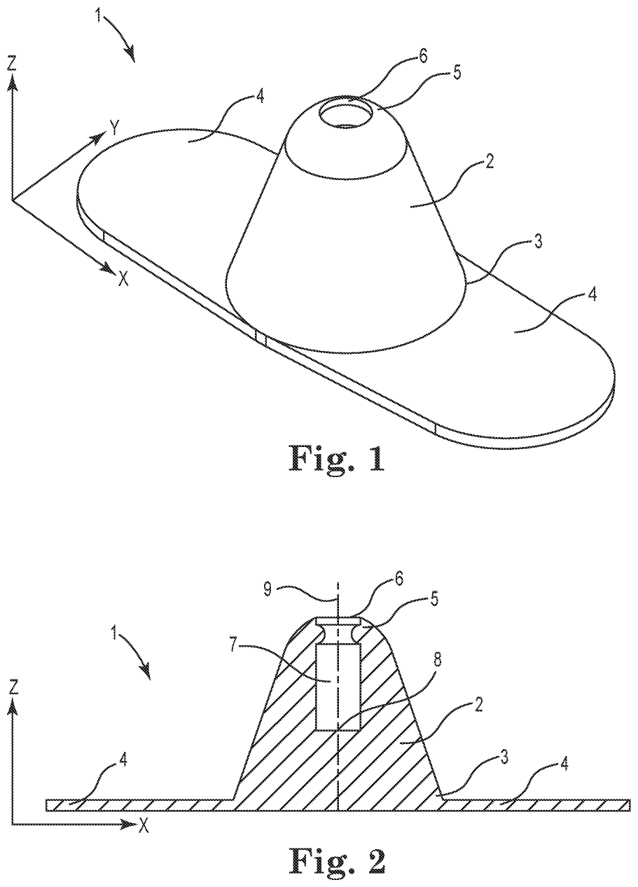

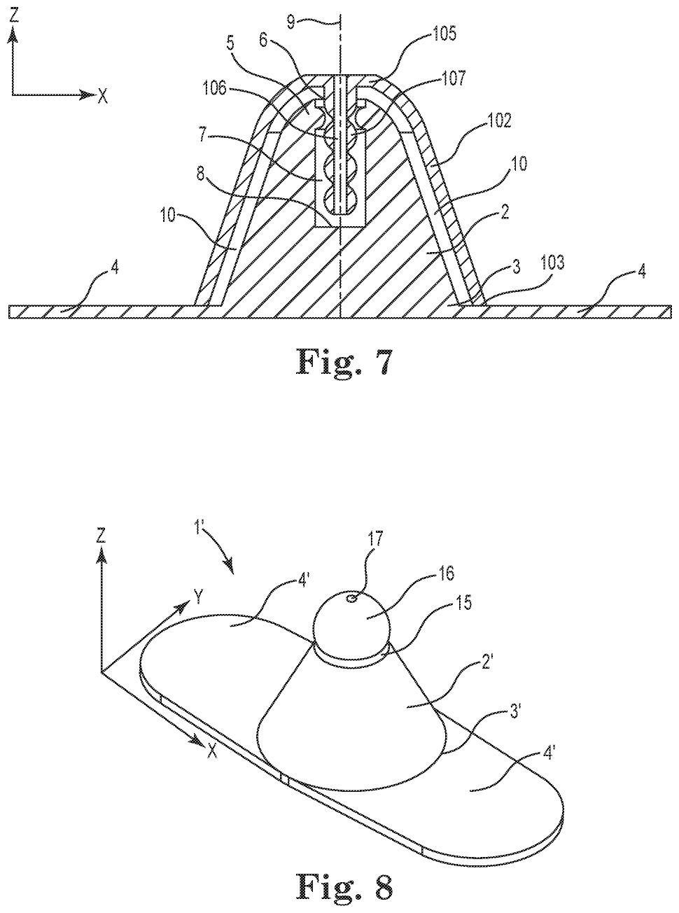

One illustrative embodiment of a vascular closure device as described herein is depicted in FIGS. 1-7. The depicted embodiment of a vascular closure device includes an inner layer 1, an outer layer 101, and an optional sealing component (e.g., a collagen or collagen-like layer) 10 (see FIG. 7) located between the inner layer 1 and the outer layer 101. FIG. 1 is a perspective view of inner layer 1 and FIG. 2 is a side view of inner layer 1 according to one embodiment. Inner layer 1 includes an intravascular tapered body 2. At least a portion of inner layer 1 is configured to be placed within a punctured blood vessel (such as, e.g., blood vessel 13′ seen in FIG. 16).

Intravascular tapered body 2 is, in one or more embodiments, tapered inward towards a first apex 5 along variable curvatures, e.g., the surface of the body 2 may not be in the form of a developable surface. In other embodiments the intravascular tapered body 2 may not be tapered inward and may instead reach toward a true point apex or conversely intravascular tapered body 2 may be a frustum shape, for example. Intravascular tapered body 2 also includes a first base 3. First base 3 is in a circular shape. In other embodiments it may be a non-circular shape, such as, e.g., a triangle, square, pentagon, hexagon, oval, etc.

Anchor 4 is attached to or detached or detachable from first base 3 and may be manufactured from the same material as intravascular tapered body 2 or from different material. Anchor 4 may be attached to first base 3 during or after production and may be attached using various means generally known (such as injection molding or adhesion, for example). Anchor 4 may also be detached from (when, e.g., delivered) but attachable to the intravascular tapered body. Intravascular tapered body 2 includes an aperture 6 proximate the first apex 5, and an interior channel 7 extending along a channel axis 9 (which, in the depicted illustrative embodiment, aligned with Z-axis of a Cartesian coordinate system provided in FIGS. 1, 2, and 7). Interior channel 7 extends through intravascular tapered body 2 along channel axis 9 away from first apex 5 towards first base 3. Further, interior channel 7 extends from aperture 6 to a first end 8. First end 8 is located between first base 3 and aperture 6. Interior channel 7 includes a constriction nearer to first apex 5 than first end 8.

Anchor 4 may have a delivery configuration and a deployed configuration. Anchor 4 is configured to be located within blood vessel when it is in the deployed configuration and, preferably, to resist removal of the inner layer 1 from a blood vessel access (puncture) site. Anchor 4 has an anchor width measured transverse to channel axis 9, and the anchor width when anchor 4 is in the deployed configuration is greater than the anchor width when anchor 4 is in the delivery configuration. Anchor 4 may include one or more extensions away from first base 3. FIGS. 1-2 illustrate an anchor 4 with two extensions. First apex 5 is configured to protrude outside of blood vessel in the deployed configuration. In one or more embodiments, a majority of the intravascular tapered body 2 is located within the blood vessel to obliterate the vascular opening and limit or prevent bleeding outside of a blood vessel when properly deployed (see, e.g., intravascular tapered body 2′ in FIG. 18).

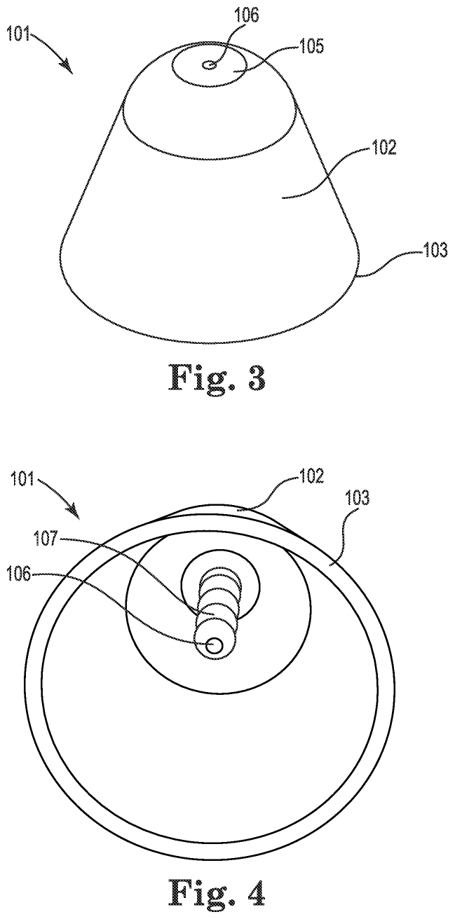

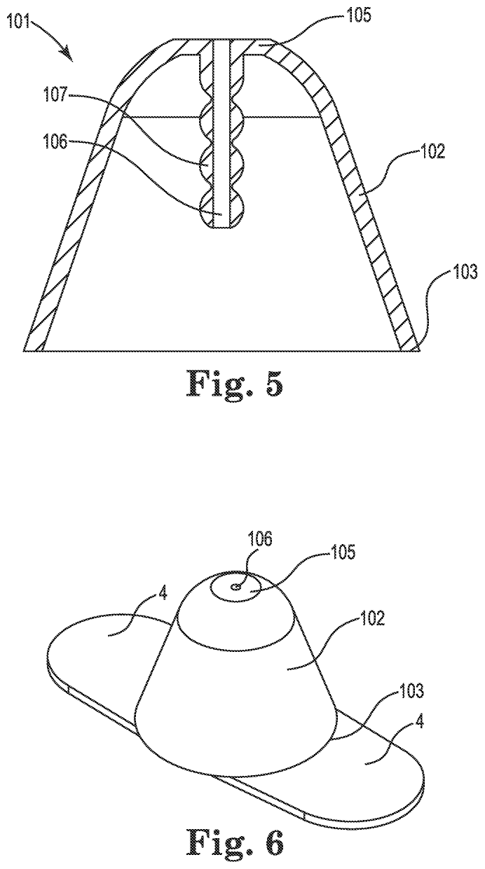

FIGS. 3-5 illustrate one illustrative embodiment of outer layer 101. FIG. 3 is a top perspective view, FIG. 4 is a bottom perspective view, and FIG. 5 is a side view of outer layer 101. Outer layer 101 includes an extravascular tapered body 102 with a second base 103 and a second apex 105. These dimensions may vary in size based on device performance to secure the pressurized arterial system and prevent blood from extravasation outside the vascular access site.

Extravascular tapered body 102 is tapered toward second apex 105 along variable curvatures, e.g., the surface of the body 102 may not be in the form of a developable surface. In other embodiments body 102 may not be tapered inward and may instead reach toward a true point apex or conversely extravascular tapered body 102 may be a frustum shape, for example. Second base 103 is in a circular shape. In other embodiments it may be a non-circular shape, such as, e.g., a triangle, square, pentagon, hexagon, oval, etc. In one or more embodiments, the shape of the interior of the extravascular tapered body 102 may be complementary to the shape of the intravascular tapered body 2 such that the intravascular tapered body 2 can be closely received within the extravascular tapered body 102. The shape of one or both of the intravascular tapered body 2 and the extravascular tapered body 102 may, in one or more embodiments, vary to accommodate the sealing component.

Outer layer 101 also includes a male locking component which includes an interior shaft 107. Interior shaft 107 extends from second apex 105 towards second base 103 to a second end located between second apex 105 and second base 103. Interior shaft 107 also includes a through-hole 106 aligned with channel axis 9 and configured to house a string (not shown) during placement of the vascular closure device in a patient. Interior shaft 107 is configured to be inserted into interior channel 7 of intravascular tapered body 2. The depicted illustrative embodiment of interior shaft 107 is composed of four bulbous components. In other embodiments, interior shaft 107 may be made up of one or more bulbous components or any other suitable shape or shapes configured to retain the extravascular tapered body 102 in place on the intravascular tapered body 2 as described herein.

FIGS. 6-7 illustrate how, in the depicted illustrative embodiment, inner layer 1 and outer layer 101 and sealing component 10 interact to form one or more embodiments of a vascular closure device as described herein. Sealing component 10 may preferably be resiliently compressible and/or deformable to facilitate closure of a vascular opening. In one or more embodiments, the sealing component 10 may be constructed of collagen or any collagen-like substance that changes in shape and/or volume based on its position depending on its delivery or deployed position and is located between intravascular tapered body 2 and at least a portion of an interior of extravascular tapered body 102. In one or more embodiments, sealing component 10 may be provided on the exterior surface of the intravascular tapered body 2 (i.e., the surface facing the interior surface of the extravascular tapered body 102) and/or on the interior surface of the extravascular tapered body 102 (i.e., the surface facing the exterior surface of the intravascular tapered body 2). Sealing component 10 and outer layer 101 may, in one or more embodiments, reduce and/or absorb any residual bleeding from the blood vessel in which the inner layer 1 is deployed. Sealing component 10 may, in one or more embodiments, be sized such that sealing component 10 extends farther from channel axis 9 than intravascular tapered body 2 and/or outer layer 101 over the surface of skin surrounding a vascular opening in which the vascular closure device is deployed. Doing so may, in one or more embodiments, improve the effectiveness of sealing the vascular opening.

Intravascular tapered body 2 is, in the depicted embodiment, located within extravascular tapered body 102 and sealing component 10 when interior shaft 107 is located inside interior channel 7 of intravascular tapered body 2. Interior shaft 107 forms an interference fit with interior channel 7 when interior shaft 107 is inserted into interior channel 7. That interference fit may, in one or more embodiments, assist in retaining the extravascular tapered body 102 in position on intravascular tapered body 2 And may maintain the intravascular tapered body 2 and the extravascular tapered body 102 in a pressurized state designed to seal the vessel opening in the blood vessel including, e.g., counteracting the intravascular pressure to prevent bleeding.

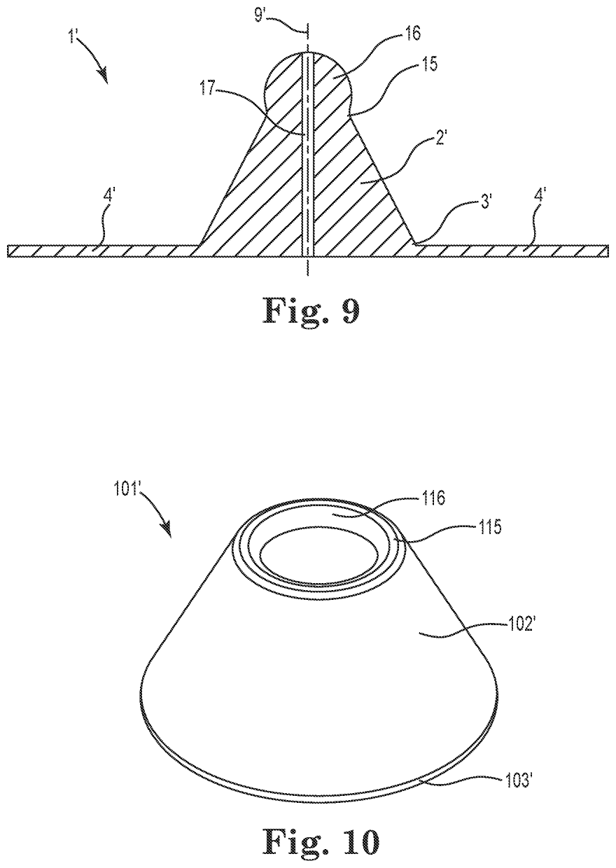

FIGS. 8-9 illustrate an inner layer 1′ according to one alternative embodiment. FIG. 8 is a perspective view and FIG. 9 is a side view of inner layer 1′. Inner layer 1′ includes an intravascular tapered body 2′. Inner layer 1′ is configured to be placed within a punctured blood vessel (not shown). Intravascular tapered body 2′ is tapered inward towards an upper end 15 along variable curvatures, e.g., the surface of the body 2′ may not be in the form of a developable surface. In other embodiments it may not be tapered inward and may instead reach toward a true point apex or conversely intravascular tapered body 2′ may be a frustum shape, for example. Intravascular tapered body 2′ also includes a first lower base 3′. First lower base 3′ is in a circular shape. In other embodiments it may be a non-circular shape, e.g., a triangle, square, pentagon, hexagon, oval, etc.

Anchor 4′ is attached to or detached or detachable from first lower base 3′ and may be manufactured from the same material as intravascular tapered body 2′ or from different material. Anchor 4′ may be attached to first lower base 3′ during or after production and may be attached using various means generally known (such as injection molding or adhesion, for example). Anchor 4′ may also be detached from the intravascular tapered body (when, e.g., delivered). Anchor 4′ may also be in a perpendicular or a parallel position in a delivery configuration or in a deployed configuration. Intravascular tapered body 2′ includes a bulbous top 16 which is attached to upper end 15, and a through-hole 17 extending along an axis 9′. Through-hole 17 extends through intravascular tapered body 2′ along axis 9′ from bulbous top 16 to first lower base 3′.

Anchor 4′ may have a delivery configuration and a deployed configuration. Anchor 4′ is configured to be located within blood vessel 13′ (not shown) when it is in the deployed configuration. Anchor 4′ has an anchor width measured transverse to axis 9′, and the anchor width when anchor 4′ is in the deployed configuration is greater than the anchor width when anchor 4′ is in the delivery configuration. Anchor 4′ may include one or more extensions away from first lower base 3′. The shape of the anchor 4′ may also be variable from a linear to elliptical, circular, or other shapes. FIGS. 1-2 illustrate an anchor 4′ with two extensions. Upper end 5′ is configured to protrude outside of blood vessel (not shown) in the deployed configuration. Anchor 4′ is configured to be located within a blood vessel 13′ when in the deployed configuration, and intravascular tapered body 2′ and bulbous top 16 are configured to protrude outside the blood vessel when anchor 4′ is in the deployed configuration in blood vessel.

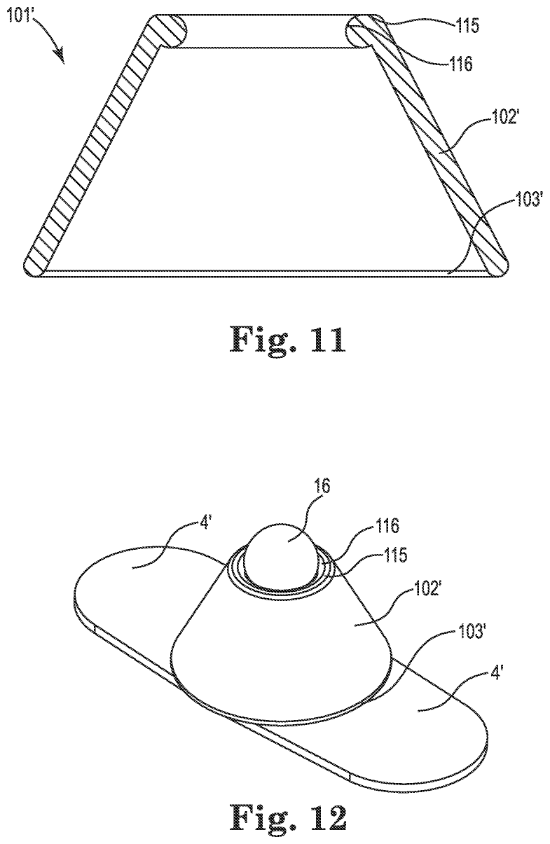

FIGS. 10-11 illustrate one embodiment of outer layer 101′ configured for use with inner layer 1′. FIG. 10 is a perspective view and FIG. 11 is a side view of outer layer 101′. Outer layer 101′ includes an extravascular tapered body 102′ with a second base 103′ and an upper aperture 115. Extravascular tapered body 102′ is tapered toward upper aperture 115 along variable curvatures, e.g., the surface of the body 102′ may not be in the form of a developable surface. In other embodiments it may not be tapered inward and may instead reach toward a true point apex or conversely extravascular tapered body 102′ may be a frustum shape, for example. Second base 103′ is in a circular shape. In other embodiments it may be a non-circular shape, e.g., a triangle, square, pentagon, hexagon, oval, etc. In one or more embodiments, the shape of the interior of the extravascular tapered body 102′ may be complementary to the shape of the intravascular tapered body 2′ such that the intravascular tapered body 2′ can be closely received within the extravascular tapered body 102′. These dimensions may vary in size based on device performance to secure the pressurized arterial system and prevent blood from extravasation outside the vascular access site. The fit between the intravascular tapered body 2′ and the extravascular tapered body 102′ may maintain the device in a pressurized state designed to seal the vessel opening in the blood vessel including, e.g., counteracting the intravascular pressure to prevent bleeding.

Outer layer 101′ may also include an optional interior shoulder 116 which includes a curved edge for better interference fit with inner layer 1′. Other embodiments may include an interior shoulder 116 with non-curved or partially curved edges to improve fit, and outer layer 101′ may maintain the outside and inside components in a pressurized configuration.

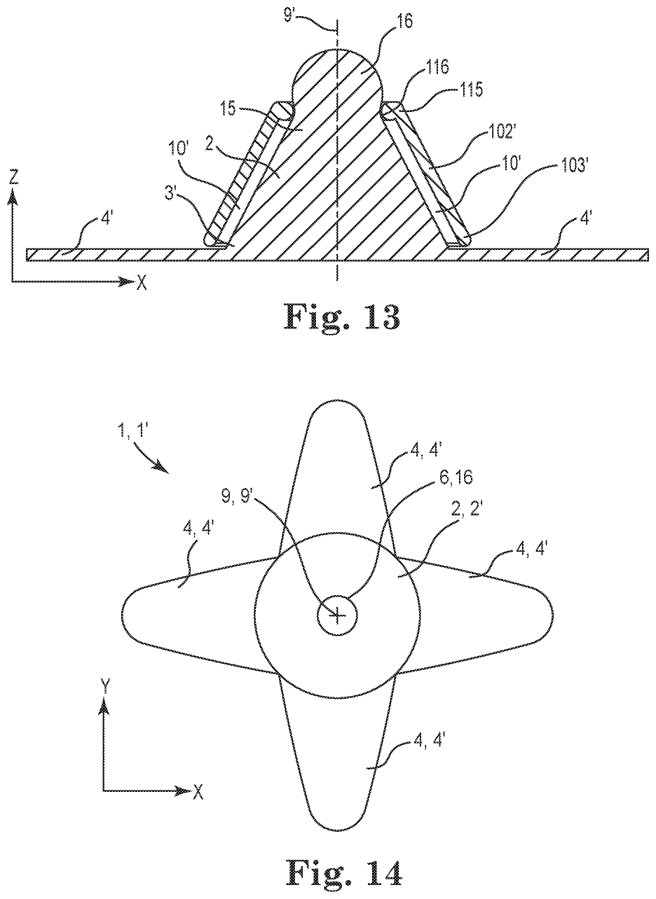

FIGS. 12-13 illustrate how inner layer 1′ and outer layer 101′ and sealing component 10′ interact to form a vascular closure device according to one embodiment. Sealing component 10′ can be made up of collagen or any collagen-like substance and is located between intravascular tapered body 2′ and at least a portion of an interior of extravascular tapered body 102′. In one or more embodiments, sealing component 10′ can be provided on the exterior surface of the intravascular tapered body 2′ (i.e., the surface facing the interior surface of the extravascular tapered body 102′) and/or on the interior surface of the extravascular tapered body 102′ (i.e., the surface facing the exterior surface of the intravascular tapered body 2′). Sealing component 10′ and outer layer 101′ are preferably able to reduce and/or absorb any residual bleeding from the blood vessel in which the inner layer 1′ is deployed. Sealing component 10′ may change in shape and volume based on its position depending on its delivery or deployed position. Both the intravascular tapered body 2′ and the extravascular tapered body 102′ will vary to accommodate the sealing component.

Bulbous top 16 forms, in the depicted illustrative embodiment, an interference fit with upper aperture 115 and with optional interior shoulder 116 when bulbous top 16 is inserted through upper aperture 115 of extravascular tapered body 102′. Intravascular tapered body 2′ is located within the extravascular tapered body 102′ such that second lower base 103′ of extravascular tapered body 102′ is located adjacent to first lower base 3′ of intravascular tapered body 2′ and upper aperture 115 of extravascular tapered body 102′ is located adjacent to upper end 15 of intravascular tapered body 2′ in the deployed configuration. In the depicted illustrative embodiment, the shape of the bulbous top 16 is a generally smooth spherical shape. In one or more alternative embodiments, the bulbous top 16 may take any suitable shape, e.g., a geodesic spherical shape, a polyhedron, etc., or any suitable configuration and position in relation to axis 9′. Bulbous top may form a cap over through-hole 17, or through-hole 17 may be closed or plugged in various other ways known to one of skill in the art., such as, e.g., a separate cap piece, etc.

FIG. 14 illustrates a top-down view of inner layer 1, 1′. In this illustrative embodiment, the anchor 4, 4′ includes four extensions or lobes. One or more alternative embodiments of anchors used in the devices described herein may include three extensions/lobes or five or more extensions/lobes. Channel axis 9/axis 9′ extend down the center of intravascular tapered body 2, 2′. Aperture 6/bulbous top 16 are also pictured top-down, and are located on the intravascular tapered body 2, 2′.

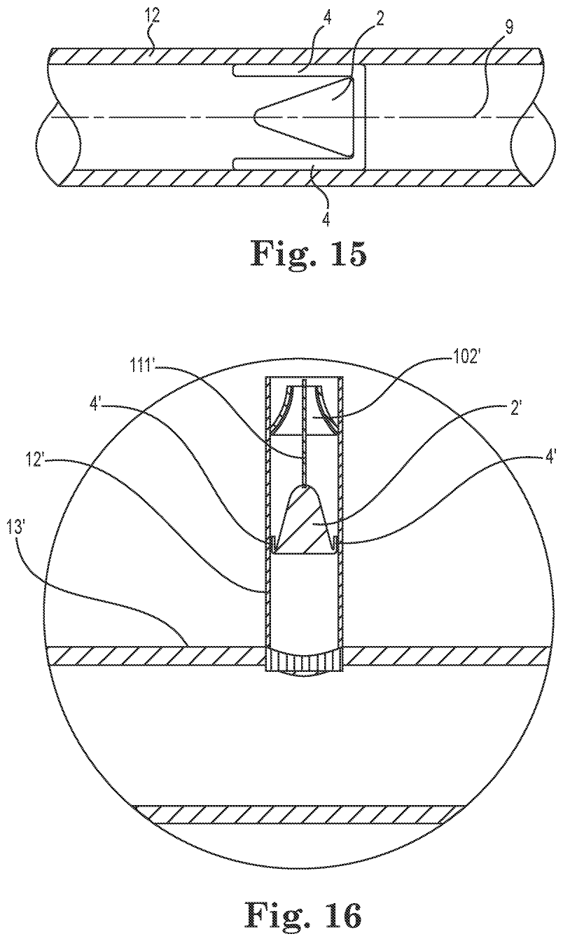

FIG. 15 illustrates one embodiment of the deployment of a vascular closure device of FIGS. 1-7 into blood vessel (not shown) through a delivery lumen of a delivery device 12 in one embodiment. Delivery device 12 may be, e.g., a cannula, catheter, sheath or other delivery device providing a delivery lumen. Deployment or delivery of the vascular closure devices through a delivery lumen may involve pushing, pulling, or a combination of pushing and pulling to move a vascular closure device through the delivery lumen using any suitable techniques or structures. Delivery of the vascular closure devices into a blood vessel as depicted in FIG. 15 can, in one or more embodiments, be similar to the delivery of another embodiment as described below. Another configuration may be where anchor 4′ is completely detached and delivered along axis 9′ of delivery device 12 before, e.g., being attached to the reminder of the vascular closure device.

FIGS. 16-18 depict one illustrative embodiment of deployment of a vascular closure device as described herein using a delivery device 12′ in the form of, e.g., a cannula, a catheter, etc.

FIG. 16 illustrates how the vascular closure device of FIGS. 8-13 is configured to be delivered into blood vessel 13′ through a delivery lumen of a delivery device 12′. In one or more embodiments, the intravascular tapered body 2′ and associated anchor 4′ (with anchor 4′ folded upward towards the apex of intravascular tapered body 2′) are advanced through the delivery lumen of delivery device 12′ using any suitable techniques and/or structures. In one or more embodiments, the intravascular tapered body 2′ and associated anchor 4′ may be advanced towards blood vessel 13′ using a plunger (not shown), fluid pressure, etc.

In one or more embodiments, a string 111′ may be provided to assist with proper deployment by, e.g., providing the ability to pull the intravascular tapered body 2′ away from the blood vessel 13′ after the anchor 4′ is deployed within the blood vessel 13′. String 111′ (e.g., suture material, etc.) may, in one or more embodiments, also serve to align the extravascular tapered body 102′ relative to the intravascular tapered body 2′ during deployment of the vascular closure device. Although a string 111′ is used in the depicted embodiment, any suitable structure capable of assisting with proper seating of the intravascular tapered body 2′ and anchor 4′ may be used in place of string, e.g., a cable, a rod, a chain etc.

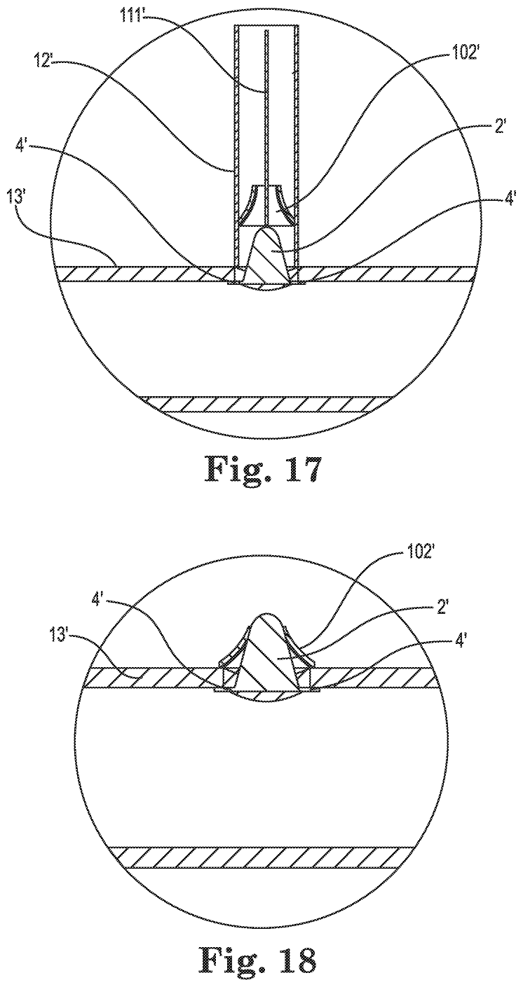

FIG. 17 illustrates how the vascular closure device of FIGS. 8-13 is placed within blood vessel 13′ during deployment. Anchor 4′ and the lower base of intravascular tapered body 2′ are advanced into blood vessel 13′ where anchor 4′ is deployed. Intravascular tapered body 2′ is pulled away from the blood vessel 13′ to ensure that the intravascular tapered body 2′ is properly seated at the puncture site of the blood vessel 13′.

FIG. 18 illustrates another step in deployment of the vascular closure device of FIGS. 8-13. The optional sealing component (not shown) may be placed over intravascular tapered body 2′. Extravascular tapered body 102′ is advanced towards and over intravascular tapered body 2′ and the optional sealing component (not shown) such that an interference or other locking fit is ensured therebetween. Such placement can be performed by using a plunger (other pushing device) to advance extravascular tapered body 102′ towards and over intravascular tapered body 2′. String 111′ may be removed after deployment of the extravascular tapered body 102′ on the intravascular tapered body 2′.

FIGS. 16-18 demonstrated simple deployment and do not show every detail of inner layer 1′, such as the bulbous top 16. Alternatively, the inner layer 1′ may be delivered on its side and perpendicular to how it is deployed for ease of delivery, and then pulled taut using, e.g., string 111′ such that the anchor 4′ is pulled against the side of blood vessel 13′ and the intravascular tapered body 2′ obliterates the vessel opening in the blood vessel and protrudes outside of blood vessel 13′.

Another illustrative embodiment of a vascular closure device 30 as described herein is depicted in FIG. 19 in which the vascular closure device is in a partially assembled state. The vascular closure device 30 is depicted during deployment in FIG. 20 in which the vascular closure device 30 is partially contained within a delivery apparatus 90 position in an access site 33 formed in a wall 32 of a blood vessel such that the distal portions of the vascular closure device 30 and the distal end of the delivery apparatus 90 are positioned within the interior volume of the blood vessel. The vascular closure device 30 is depicted in FIGS. 21-22 in its deployed state outside of the blood vessel depicted in FIG. 20 to facilitate discussion of the various components of the vascular closure device.

The illustrative embodiment of vascular closure device 30 as depicted in FIGS. 19-20 includes an outer component 40, and inner component 50, a locking element 60, seal component 70, and tensioning element 80. The outer component 40 and the inner component 50 work together to form an expandable body configured to occupy an access site 33 in the wall 32 of a blood vessel. The tubular seal 74 of seal component 70 as depicted in FIGS. 19 and 21-22 is depicted in broken lines to allow for visualization of the features inside the tubular seal 74.

In the depicted illustrative embodiment of vascular closure device 30 the inner component 50 is in the form of a cone shaped inner component 50 extending from a base 52 to an apex 54 along a device axis 31 extending through the various components of the vascular closure device 30.

In the depicted illustrative embodiment of vascular closure device 30 the outer component 40 defines a passageway 43 extending along the device axis 31 from an anchor portion proximate the base 52 of the inner component 50 to a collar 42 proximate the apex 54 of the inner component 50 such that, when assembled, the apex 52 of the inner component 50 is closer to the collar 42 of the outer component 40 than the anchor portion of the outer component 40.

In the depicted illustrative embodiment of outer component 40, the anchor portion is defined by a plurality of anchor feet 46 found at the bottom ends of ribs 44 that extend away from the collar 42 along the device axis 31. When assembled with the inner component 50, the ribs may be described as extending from the collar 42 towards the base 52 of the inner component 50. The anchor feet 46 may be described as extending away from the device axis 31 such that the radial dimension (relative to device axis 31) of the vascular closure device 30 in its assembled state (as depicted in, for example, FIGS. 21-22) is largest within the anchor portion as defined by the feet 46.

The ribs 44 of the outer component 40 are separated to facilitate expansion of the outer component as the inner component 50 is advanced into the passageway 43 of the outer component 40. Although depicted as separated in the illustrative embodiment, one or more alternative embodiments may include an outer component in which the portion of the outer component between the anchor portion 46 and the collar 42 is expandable without requiring separate and distinct ribs 44 as depicted in the illustrative embodiment. For example, the portion of the outer component 40 between the anchor portion 46 and the collar 42 may be formed of an expandable material (e.g., silicone, thermoplastic elastomers, etc.), a combination of two or more materials one or more of which may be expandable, etc.

The inner component 50 as depicted is in the form of a truncated cone shaped body formed as a composite of right circular cones having different heights such that the outer wall of the inner component 50 converge towards the device axis 31 at different angles to, for example, facilitate assembly of the inner component 50 with the outer component 40 of the illustrative embodiment of vascular closure device 30. It should be understood that the inner component 50 may, however, take any suitable shape capable of expanding the outer component 40 as the inner component 50 advances through the passageway 43 of the outer component 40. For example, the inner component 50 may be constructed with a base having a pentagonal, hexagonal, etc. shape that narrows when moving along the device axis 31 from the base through the collar of the outer component.

The illustrative embodiment of vascular closure device 30 also includes a locking element 60 attached to the inner component 50 proximate the apex 54 of the inner component 50. In the depicted illustrative embodiment, the locking element 60 is configured to form a mechanical interlock with the collar 42 of the outer component 40 to resist movement of the inner component 50 out of the passageway 43 of the outer component 40. In the depicted illustrative embodiment, that mechanical interlock between the locking element 60 and the collar 42 is in the form of a size differential in which the bottom face or shoulder of the locking element 60 is larger than the opening in the collar 42 through which the locking element 60 moves during advancement of the inner component 50 into the passageway 43 of the outer component 40. That size differential resists or prevents movement of the locking element 60 back through the opening in the collar 42. Other mechanical interlocking structures may be provided between collar 42 and locking element 60 such as, e.g., zip tie like structures, Chinese finger cuff structures, etc.

The illustrative embodiment of vascular closure device 30 also includes a tensioning element 80 that may be used to provide a tension force to draw the locking element 60 through the opening in collar 42 and into a mechanical interlock with the collar 42 of the outer component 40 as well as advance the inner component 50 into the passageway 43 of outer component 40.

In the depicted embodiment, tensioning element 80 also connects the locking element 60 to the inner component 50, preferably at the apex 54 of the inner component 50. In one or more embodiments, the tensioning element 80 may be in tension between the locking element 60 and the inner component 50 when the locking element 60 is in a mechanical interlock with the collar 42 of the outer component 40 as described herein.

In one or more embodiments, the tensioning element 80 may be in the form of suture material, although any suitable construction capable of providing a tension force as described herein may be used in place of suture material, e.g., wires, cables, rods, etc.

To facilitate assembly of the illustrative embodiment of vascular closure device 30, the collar 42 may include an assembly slot 48 configured to allow passage of the tensioning element 80 into the opening of the collar 42 during assembly of the inner component 50 and the outer component 40. The assembly slot 48 may also provide additional compliance to the collar 42 to allow the opening in collar 42 to flex as the locking element 60 is advanced through the collar 42 as described herein.

The illustrative embodiment of vascular closure device 30 also includes a seal component 70 that includes a seal ring 72 and a tubular seal 74. In one or more embodiments, the seal ring 72 is attached to the tubular seal 74. The tubular seal 74 may, in one or more embodiments, preferably be expandable and deformable (as depicted in, for example, FIGS. 21-22) to provide a seal around the access site 33 in the wall 32 of blood vessel 34 to, e.g., limit or prevent leakage around the periphery of the vascular closure device 30, limit or prevent dirt or contaminants from entering the blood vessel through the access site 33, etc. In one or more embodiments, the tubular seal 74 may be in the form of a collagen containing body to both provide a seal and promote healing of the access site.

The seal ring 72 of the seal component 70 may, in one or more embodiments, be configured to form a second mechanical interlock with the collar 42 of the outer component 40 when the tubular seal 74 is positioned over the outer component 40 as seen in, for example, FIGS. 21-22. The mechanical interlock between the seal ring 72 and the collar 42 of the outer component 40 may, in one or more embodiments, resist movement of the seal ring 72 away from the anchor portion 46 of the outer component 40 (and also, therefore, the base 52 of the inner component 50 located within passageway 43 of outer component 40).

The mechanical interlock between the seal ring 72 and the collar 42 of the outer component 40 may be formed between a bottom face of the collar 42 (i.e., a surface of the collar 42 facing towards the anchor portion (e.g., feet 46) of the outer component 40) and the upper surface of the seal ring 72 (i.e., the surface facing away from the tubular seal 74 of the seal component 70). In particular, the bottom face of the collar 42 interferes with the upper surface of the seal ring 72 such that movement of the seal ring 72 away from the anchor portion 46 of the outer component as well as away from the inner component 50 is resisted by mechanical interference. In one or more embodiments, the collar 42 of the outer component 40 may be described as being positioned between the locking element 60 and the seal ring 72 of seal component 70 when the locking element 60 forms a mechanical interlock with the collar 42 and the seal ring 72 forms a mechanical interlock with the collar 42.

In one or more embodiments, the seal ring 72, when seated on the collar 42 as depicted in, e.g., FIGS. 21-22, may also provide additional support to resist expansion of the opening in collar 42 through which locking element 60 moves during deployment of the vascular closure device 30.

One illustrative embodiment of a delivery apparatus 90 that can be used to deploy the illustrative embodiment of vascular closure device 30 and one illustrative embodiment of deployment of the vascular closure device 30 may be described with reference to FIGS. 20 and 23-26. The illustrative embodiment of delivery apparatus 90 includes a series of concentrically arranged tubular members used to counteract the tension forces provided by the tensioning element 80 during deployment of the vascular closure device 30 as described herein. As a result, the tubular members may preferably have sufficient mechanical strength and compression along the device axis 31 to resist the tension forces supplied using the tensioning element 80 as described herein.

In particular, the delivery apparatus 90 includes an outer sheath 92, a seal sheath 94 located within the outer sheath 92, and collar stabilizer 96 located within the seal sheath 94. Although described as an outer sheath 92, the outer sheath 92 may or may not be used to advance the vascular closure device 30 and distal end of the delivery apparatus 90 through the access site 33 in the wall 32 of a blood vessel 34. For example, a conventional introducer sheath or other device may be used to provide a lumen through which the delivery apparatus 90 and vascular closure device 30 can be advanced to position the delivery apparatus 90 and the vascular closure device 30 within a blood vessel as depicted in, e.g., FIG. 20.

With reference to FIG. 20, with the outer sheath 92 positioned through the access site 33 in wall 32 of blood vessel 34 and the components of the vascular closure device 30 at least partially or completely located within the blood vessel 34, assembly and positioning of the vascular closure device 30 in the access site 33 can begin.

With reference to FIGS. 20 and 24, the inner component 50 of the vascular closure device 30 is drawn into the passageway 43 and outer component 40 to expand the outer component 40 radially and also position the locking element 60 above the collar 42 to form a mechanical interlock between the locking element 60 and the collar 42 of the outer component 40. These changes can be seen by comparing the positions of the inner component 50 relative to the outer component 40 in FIGS. 20 and 24 as well as the positions of the locking element 60 relative to the outer component 40 in FIGS. 20 and 24.

In particular, movement of the inner component 50 and the locking element 60 is provided by a tension force on both the locking element 60 and the inner component 50 through tensioning element 80 as depicted by arrow 81 in FIG. 24 as well as a corresponding compressive force provided on the collar 42 of the outer component 40 using the collar stabilizer 96 as depicted by arrows 97 in FIG. 24. The tension and compression forces provided by, respectively, tensioning element 80 and collar stabilizer 96, provide for relative movement of the inner component 50 locking element 60 and outer component 40 to achieve the positions depicted in FIG. 24 while the outer sheath 92 remains positioned through or across the access site 33.

Also depicted with respect to FIGS. 20 and 24, are expansion of the anchor portion of outer component 40 (as defined by the anchor feet 46 of outer component 40) when the inner component 50 is advanced into the passageway 43 of outer component 40 and locking element 60 is moved into its interlocking configuration with collar 42 of outer component 40.

Expansion of the anchor portion of outer component 40 is preferably sufficient to retain the base 52 of the inner component 50 and the anchor portion 46 of the outer component 40 within the interior volume of the blood vessel 34 such that withdrawal of the outer sheath 92 relative to the vascular closure device 30 results in seating of the anchor portion 46 around the interior perimeter of the access site 33 through wall 32 of blood vessel 34 as seen in FIG. 25.

With the vascular closure device 30 seated in access site 33, the outer sheath 92 of the delivery apparatus 90 may be further withdrawn from the vascular closure device 30 as depicted by arrows 91 in FIG. 25. Further withdrawal of the outer sheath 92 can be used to expose the seal component 70 located within outer sheath 92 to facilitate deployment of the seal component 70 to close the access site 33.

With reference to FIG. 26 in which the outer sheath 92 has been withdrawn from the vascular closure device 30, advancement of seal sheath 94 in the direction of arrows 95 towards the access site 33 forces advancement of the seal ring 72 over the collar 42 of the outer component 40 to form a mechanical interlock between the seal ring 72 and the collar 42 as described herein. Advancement of the seal ring 72 causes the tubular seal 74 of the seal component 70 to compress and deform around the vascular closure device 30 in access site 33 2 seal the access site 33 as described herein.

A tension force (see arrow 81 in FIG. 26) may be provided on the outer component 40 locking element 60 and inner component 50 using tensioning element 80 during advancement of the seal ring 72 by the seal sheath 94 to maintain proper positioning of the vascular closure device within the access site 33 while the seal component 70 is deployed. Although not required, proper positioning of the outer component 40 during this process may also be assisted by the collar stabilizer 96 acting on the outer component 40.

With the vascular closure device properly seated and sealed within the access site 33, the tubular elements of the delivery apparatus 90 may be removed and, if desired, the tensioning element 80 may also be severed above the locking element 60.

Another illustrative embodiment of a vascular closure device as described herein is depicted in FIGS. 27-30. The vascular closure device 130 as depicted in FIG. 27 is in its deployed state outside of a blood vessel in a manner similar to the vascular closure device 30 as depicted in FIG. 21. FIG. 28 is a cross-sectional view in which the deployed vascular closure device is located within an access site 133 in the wall 132 of a blood vessel 134 in a manner similar to the vascular closure device 30 as depicted in FIG. 26. FIGS. 29-30 depict the vascular closure device 130 in a partially assembled state.

The illustrative embodiment of vascular closure device 130 as depicted in FIGS. 27-30 includes an outer component 140, and inner component 150, a locking element 160, seal component 170, and tensioning element 180. The outer component 140 and the inner component 150 work together to form an expandable body configured to occupy an access site 133 in the wall 132 of a blood vessel 134. The tubular seal 174 of the seal component as depicted in FIGS. 27 and 29 is depicted in broken lines to allow for visualization of the features inside the tubular seal 174.

In the depicted illustrative embodiment of vascular closure device 130 the inner component 150 is in the form of a cone shaped inner component 150 extending from a base 152 to an apex 154 along a device axis 131 extending through the various components of the vascular closure device 130.

In the depicted illustrative embodiment of vascular closure device 130 the outer component 140 defines a passageway 143 (see FIG. 30) extending along the device axis 131 from an anchor portion proximate the base 152 of the inner component 150 to a collar 142 proximate the apex 154 of the inner component 150 such that, when assembled, the apex 152 of the inner component 150 is closer to the collar 142 of the outer component 140 than the anchor portion of the outer component 140.

In the depicted illustrative embodiment of outer component 140, the anchor portion is defined by a plurality of anchor feet 146 found at the bottom ends of ribs 144 that extend away from the collar 142 along the device axis 131. When assembled with the inner component 150, the ribs 144 may be described as extending from the collar 142 towards the base 152 of the inner component 150. The anchor feet 146 may be described as extending away from the device axis 131 such that the radial dimension (relative to device axis 131) of the vascular closure device 130 in its assembled state (as seen in, for example, FIG. 27) is largest within the anchor portion as defined by the feet 146.

The ribs 144 of the outer component 140 are separated to facilitate expansion of the outer component as the inner component 150 is advanced into the passageway 143 of the outer component 140. Although depicted as separated in the illustrative embodiment, one or more alternative embodiments may include an outer component in which the portion of the outer component between the anchor portion 146 and the collar 142 is expandable without requiring separate and distinct ribs 144 as depicted in the illustrative embodiment. For example, the portion of the outer component 140 between the anchor portion 146 and the collar 142 may be formed of an expandable material (e.g., silicone, thermoplastic elastomers, etc.), a combination of two or more materials one or more of which may be expandable, etc.

The inner component 150 as depicted is in the form of a truncated cone shaped body formed as a composite of right circular cones having different heights such that the outer wall of the inner component 150 converge towards the device axis 131 at different angles to, for example, facilitate assembly of the inner component 150 with the outer component 140 of the illustrative embodiment of vascular closure device 130. It should be understood that the inner component 150 may, however, take any suitable shape capable of expanding the outer component 140 as the inner component 50 advances through the passageway 143 of the outer component 140. For example, the inner component 150 may be constructed with a base having a pentagonal, hexagonal, etc. shape that narrows when moving along the device axis 131 from the base through the collar of the outer component.

The illustrative embodiment of vascular closure device 130 also includes a locking element 160 attached to the inner component 150. Unlike the locking element 60 attached to inner component 50 described above in connection with the illustrative embodiment of vascular closure device 30, the locking element 160 defines the apex 154 of the inner component 150. In the depicted embodiment, the inner component 150 may be described as including a neck 162 located between the locking element 160 and a remainder of the inner component 150. When seated in the collar 142 as seen in, e.g., FIG. 28, the neck 162 may be compressed within the opening of the collar 142 and or may be in tension between the upper surface of the collar 142 and the ribs 144 of the outer component 140.

In the depicted illustrative embodiment, the locking element 160 is configured to form a mechanical interlock with the collar 142 of the outer component 140 to resist movement of the inner component 150 out of the passageway 143 of the outer component 140. In the depicted illustrative embodiment, that mechanical interlock between the locking element 160 and the collar 142 is in the form of a size differential in which the bottom face or shoulder of the locking element 160 is larger than the opening in the collar 142 through which the locking element 160 moves during advancement of the inner component 150 into the passageway 143 of the outer component 140. That size differential resists or prevents movement of the locking element 160 back through the opening in the collar 142. Other mechanical interlocking structures may be provided between collar 142 and locking element 160 such as, e.g., zip tie-like structures, Chinese finger cuff structures, etc.

The illustrative embodiment of vascular closure device 130 also includes a tensioning element 180 that may be used to provide a tension force to draw the locking element 160 through the opening in collar 142 and into a mechanical interlock with the collar 142 of the outer component 140 as well as advance the inner component 150 into the passageway 143 of outer component 140.

In one or more embodiments, the tensioning element 180 may be in the form of suture material, although any suitable construction capable of providing a tension force as described herein may be used in place of suture material, e.g., wires, cables, rods, etc.

To facilitate assembly of the illustrative embodiment of vascular closure device 130, the collar 142 may include an assembly slot 148 configured to allow passage of the tensioning element 180 into the opening of the collar 142 during assembly of the inner component 150 and the outer component 140. To prevent unwanted removal of the tensioning element 180 from the assembly slot 148, the assembly slot 148 may include a circumferential portion 149 extending around a portion of the perimeter of the collar 142. The assembly slot 148 may, in one or more embodiments, also provide additional compliance to the collar 142 to allow the opening in collar 142 to flex as the locking element 160 is advanced through the collar 142 as described herein.

The illustrative embodiment of vascular closure device 130 also includes a seal component 170 that includes a seal ring 172 and a tubular seal 174. In one or more embodiments, the seal ring 172 is attached to the tubular seal 174. The tubular seal 174 may, in one or more embodiments, preferably be expandable and deformable (as depicted in, for example, FIGS. 27-28) to provide a seal around the access site 133 in the wall 132 of blood vessel 134 to, e.g., limit or prevent leakage around the periphery of the vascular closure device 130, limit or prevent dirt or contaminants from entering the blood vessel through the access site 133, etc. In one or more embodiments, the tubular seal 174 may be in the form of a collagen containing body to both provide a seal and promote healing of the access site.

The seal ring 172 of the seal component 170 may, in one or more embodiments, be configured to form a second mechanical interlock with the collar 142 of the outer component 140 when the tubular seal 174 is positioned over the outer component 140 as seen in, for example, FIGS. 27-28. The mechanical interlock between the seal ring 172 and the collar 142 of the outer component 140 may, in one or more embodiments, resist movement of the seal ring 172 away from the anchor portion 146 of the outer component 140 (and also, therefore, the base 152 of the inner component 150 located within passageway 143 of outer component 140).

The mechanical interlock between the seal ring 172 and the collar 142 of the outer component 140 may be formed between a bottom face of the collar 142 (i.e., a surface of the collar 142 facing towards the anchor portion (e.g., feet 146) of the outer component 140) and the upper surface of the seal ring 172 (i.e., the surface facing away from the tubular seal 174 of the seal component 170). In particular, the bottom face of the collar 142 interferes with the upper surface of the seal ring 172 such that movement of the seal ring 172 away from the anchor portion of the outer component 140 as well as away from the inner component 150 is resisted by mechanical interference. In one or more embodiments, the collar 142 of the outer component 140 may be described as being positioned between the locking element 160 and the seal ring 172 of seal component 170 when the locking element 160 forms a mechanical interlock with the collar 142 and the seal ring 172 forms a mechanical interlock with the collar 142.

In one or more embodiments, the seal ring 172, when seated on the collar 142 as depicted in, e.g., FIGS. 27-28, may also provide additional support to resist expansion of the opening in collar 142 through which locking element 160 moves during deployment of the vascular closure device 130.

One illustrative embodiment of a deployment actuation apparatus that can be used to deploy the vascular closure devices described herein is depicted in FIG. 31. The depicted deployment actuation apparatus 200 is located at the proximal end of a delivery apparatus 290 defining the device axis 231 of a vascular closure device located at the end of the deployment apparatus 290. The tension forces delivered through a tensioning element as described herein along with the compression and/or stabilization forces delivered through the tubular members of the delivery apparatus can be supplied through rotary motion of collars 202 and 204 of the deployment actuation apparatus 200. Although the depicted illustrative embodiment uses rotary motion of collars 202 and 204 for actuation, any other suitable mechanisms could be used in place of and/or in addition to rotary motion including, but not limited to, trigger mechanisms, pushbutton mechanisms, lever mechanisms, slide mechanisms, etc.

Although not specifically described herein, the components of the vascular closure devices may be constructed of any suitable materials and/or combinations of materials capable of performing the functions described herein. In one or more embodiments, suitable materials may include, e.g., medical-grade silicone, absorbable materials, collagen-basing filling materials, fibrin-based materials, polyethylene glycol, polylactic acid (PLA), synthetic absorbable polymers, non-absorbable materials, metal alloys, metals (e.g., cobalt, titanium, platinum, etc.), etc. One or more of the components may be coated with materials known to reduce the risk of clotting to prevent thromboembolic complications.

The complete disclosure of the patents, patent documents, and publications cited in the Background, the Detailed Description of Illustrative Embodiments, and elsewhere herein are incorporated by reference in their entirety as if each were individually incorporated.

Illustrative embodiments of this invention are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

What is claimed is:

1. A vascular closure device comprising:
a cone-shaped inner component extending from a base to an apex along a device axis extending through the base and the apex;
an outer component defining a passageway extending along the device axis from an anchor portion proximate the base of the inner component to a collar proximate the apex of the inner component, wherein the anchor portion of the outer component is configured to expand radially relative to the device axis when the inner component advances through the passageway of the outer component; and
a locking element attached to the inner component proximate the apex of the inner component, wherein the locking element is configured to form a mechanical interlock with the collar of the outer component to resist movement of the inner component out of the passageway of the outer component.

2. A device according to claim 1, wherein the outer component comprises a plurality of ribs extending from the collar towards the base of the inner component and wherein the anchor portion comprises a plurality of anchor feet extending away from the device axis, wherein each rib of the plurality of ribs comprises one anchor foot of the plurality of anchor feet, wherein the plurality of anchor feet collectively define the anchor portion of the outer component.

3. A device according to claim 1, wherein the device comprises a tensioning element attached to the locking element and the inner component, the tensioning element extending through the collar of the outer component, and the tensioning element configured to draw the locking element through the collar when a tension force is applied to the locking element through the tensioning element.

4. A device according to claim 3, wherein the collar comprises an assembly slot configured to allow passage of the tensioning element during assembly of the inner component and the outer component.

5. A device according to claim 3, wherein the tensioning element comprises suture material.

6. A device according to claim 1, wherein the locking element defines the apex of the inner component.

7. A device according to claim 6, wherein the inner component comprises a neck located between the locking element a remainder of the inner component.

8. A device according to claim 1, wherein the locking element is attached to the apex of the inner component by a tensioning element extending from the apex of the inner component and through the locking element and the collar of the outer component, wherein a tension force applied to the tensioning element draws the locking element into the mechanical interlock with the collar of the outer component.

9. A device according to claim 8, wherein the apex of the inner component is spaced apart from the locking element such that the collar of the outer component is positioned between the locking element and the apex of the inner component.

10. A device according to claim 9, wherein the tensioning element is in tension when the locking element is in the mechanical interlock with the collar of the outer component.

11. A device according to claim 1, the device comprising a seal component that comprises a seal ring attached to a tubular seal, wherein the seal ring is configured forms a second mechanical interlock with the collar when the tubular seal is positioned over the outer component, wherein the second mechanical interlock between the seal ring and the collar resists movement of the seal ring away from the anchor portion of the outer component.

12. A device according to claim 11, wherein the collar of the outer component is positioned between the locking element and the seal ring when the locking element forms the mechanical interlock with the collar and the seal ring forms the second mechanical interlock with the collar.

13. A device according to claim 11, wherein the tubular seal conforms to an external shape of the outer component when the seal ring forms the second mechanical interlock with the collar.

14. A vascular closure device comprising:
a cone-shaped inner component extending from a base to an apex along a device axis extending through the base and the apex;
an outer component defining a passageway extending along the device axis from an anchor portion proximate the base of the inner component to a collar proximate the apex of the inner component, wherein the anchor portion of the outer component is configured to expand radially relative to the device axis when the inner component advances through the passageway of the outer component;
a locking element attached to the inner component proximate the apex of the inner component, wherein the locking element is configured to form a mechanical interlock with the collar of the outer component to resist movement of the inner component out of the passageway of the outer component;
a tensioning element attached to the locking element and the inner component, the tensioning element extending through the collar of the outer component, the tensioning element configured to draw the locking element through the collar when a tension force is applied to the locking element through the tensioning element; and
a seal component that comprises a seal ring attached to a tubular seal, wherein the seal ring is configured forms a second mechanical interlock with the collar when the tubular seal is positioned over the outer component, wherein the second mechanical interlock between the seal ring and the collar resists movement of the seal ring away from the anchor portion of the outer component.

15. A device according to claim 14, wherein the locking element is attached to the apex of the inner component by a tensioning element extending from the apex of the inner component and through the locking element and the collar of the outer component, wherein a tension force applied to the tensioning element draws the locking element into the mechanical interlock with the collar of the outer component, wherein the apex of the inner component is spaced apart from the locking element such that the collar of the outer component is positioned between the locking element and the apex of the inner component; and wherein the tensioning element is in tension when the locking element is in the mechanical interlock with the collar of the outer component.

16. A device according to claim 14, wherein the collar of the outer component is positioned between the locking element and the seal ring when the locking element forms the mechanical interlock with the collar and the seal ring forms the second mechanical interlock with the collar.

17. A method of implanting a vascular closure device in a vascular access site, the method comprising:
delivering a vascular closure device into a blood vessel through an access site, the vascular closure device comprising:
a cone-shaped inner component extending from a base to an apex along a device axis extending through the base and the apex,
an outer component defining a passageway extending along the device axis from an anchor portion proximate the base of the inner component to a collar proximate the apex of the inner component, and
a locking element attached to the inner component proximate the apex of the inner component;
retracting the collar of the outer component out of the blood vessel through the access site;
advancing the locking element and the inner component through the passageway of the outer component, wherein the inner component radially expands the anchor portion of the outer component relative to the device axis, wherein the anchor portion of the outer component is retained in the blood vessel; and
mechanically interlocking the locking element with the collar of the outer component to resist movement of the inner component out of the passageway of the outer component.

18. A method according to claim 17, wherein advancing the locking element and the inner component comprises applying a tension force to a tensioning element attached to the locking element and the inner component, wherein the tensioning element extends through the collar of the outer component.

19. A method according to claim 17, the method further comprising:
advancing a seal ring and a tubular seal of a seal component towards the anchor portion of the outer component along the device axis, wherein the tubular seal deforms to form a seal around the outer component at the access site; and
mechanically interlocking the seal ring with the collar of the outer component resists movement of the seal ring and the tubular seal away from the anchor portion of the outer component.

20. A method according to claim 17, wherein advancing the locking element and the inner component comprises applying a tension force to a tensioning element attached to the locking element and the inner component, wherein the tensioning element extends through the collar of the outer component, and wherein the method further comprises:
advancing a seal ring and a tubular seal of a seal component towards the anchor portion of the outer component along the device axis, wherein the tubular seal deforms to form a seal around the outer component at the access site; and
mechanically interlocking the seal ring with the collar of the outer component to resist movement of the seal ring and the tubular seal away from the anchor portion of the outer component, wherein mechanically interlocking the seal ring with the collar of the outer component is performed after mechanically interlocking the locking element with the collar of the outer component.
